(19) United States
(12) Patent Application Publication
(10) Pub. No.: US 2010/0185115 A1
(43) Pub. Date: Jul. 22, 2010

(54) APPARATUS FOR EVOKING AND RECORDING BIO-POTENTIALS

(75) Inventors: Elvir Causevic, St. Louis, MO (US); Christian Christiansen, Chesterfield, MO (US); Eldar Causevic, Ellisville, MO (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Everest Biomedical Instruments, Co.,, St. Louis, MO (US)

(21) Appl. No.: 12/710,603

(22) Filed: Feb. 23, 2010

Related U.S. Application Data
(62) Division of application No. 10/545,221, filed on Apr. 7, 2006.

(51) Int. Cl. A61B 5/0484

(52) U.S. Cl. 600/544

(57) ABSTRACT

Apparatus (10) for evoking and recording bio-potentials from a human subject and methods of use are described. The apparatus (10) includes a flexible member (18) with a layer of conductive material (12) disposed thereon for contacting a skin surface on the human subject. The dimensions and shape of the flexible member (18) are adapted for conforming contact between the conductive material (12) and the skin surface. A stimulus delivery element (28) is coupled to the flexible member (18) for delivering a sensory stimulus to the subject to evoke bio-potentials, which are detected and received through the layer of conductive material (12).

### TECHNICAL FIELD

The present invention relates in general to electrode apparatus and methods for measuring bio-potentials in human individuals, and more particularly to apparatus including means for providing stimuli and methods of using the apparatus for measuring evoked bio-potentials in human individuals.

### BACKGROUND ART

When multiple nerve cells or muscle cells depolarize simultaneously or sequentially, they generate a bio-potential that can be detected as an electrical signal by an externally positioned electrical circuit. External electrical circuits have long been used to measure such relatively small but measurable bio-potentials. The electrocardiogram (ECG or EKG), electromyogram (EMG), electroencephalogram (EEG), and Auditory Evoked Potentials, (AEP) are examples of systems and methods using such circuits to monitor, respectively, cardiac contractions, muscle contractions and brain cell activity.

Electrode apparatus for recording bio-potentials, for example for EEG biofeedback applications, include a minimum of one pair of electrodes, and a third electrode as the ground electrode. The pair of electrodes, including an “active” electrode and an “indifferent” electrode, record one channel of EEG signal. The active electrode is typically located on the head near a brain area being monitored, the indifferent electrode is located on the head, on an ear, or on the mastoid bone behind an ear, and the ground electrode is typically placed on the forehead or on an ear, but can be placed almost anywhere. Additional recording channels can be added as desired by adding additional electrodes.

Known electrodes and related apparatus for recording bio-potentials such as EEG's are disposable electrodes such as, but not limited to, disposable self-adhesive electrodes, ear clip electrodes, disc electrodes, needle electrodes and saline-based electrodes. With all types of electrodes, a key factor in obtaining accurate and relatively noiseless bio-potential recordings is maintaining adequate contact between the electrode and the skin, because bio-potentials are typically relatively small, i.e. less than about 20 mV, and the recordings are highly susceptible to noise and artifacts.

While the methods of ensuring proper electrical contact between the electrode and the skin vary somewhat with the type of electrode being used, the skin usually must be prepared by cleaning with alcohol and abrading with an electrode preparation gel. The steps of cleaning and abrading may be repeated several times for ideal surface preparation. Skin contact is established using an ear clip electrode with a metal clip that fastens to the outer ear, a self-adhesive disc electrode that adheres directly to an area of skin, or with a disc electrode having a cup that is filled with an electrode paste. These types of electrodes are unsuitable for use in areas with much hair, such as on the scalp, and generally provide electrical contact that is not very robust or long lasting, which affects the quality and duration of recordings that can be obtained.

Needle-type electrodes generally provide better and more long-lasting contact, and can be used on the scalp, but involves tedious, uncomfortable and costly procedure to secure contact. To utilize a needle-type electrode, the hair must be parted to reveal skin, a colloidin-treated gauze layer secured over the electrode, electrode gel injected with a hypodermic needle through a hole in an electrode cup, and finally the skin abraded with the blunt end of the needle. Headband-type and hat-style electrode connectors are known, in which electrodes, such as those described above, are coupled to a hat, or to a headband made of an elastomeric material that fits around the crown of the head, holding cup or disc-type electrodes in place across the forehead of the subject. Headband-type electrode connectors are typically used for recording signals from the frontal areas of the brain, and are less useful for recording from other areas of the brain because of the relatively poor signal quality that results. In addition, headband connectors still require careful skin preparation.

Saline-based electrodes are also known, in which salt water is used to maintain the electrical connection between electrode and skin, instead of electrode gel. An electrode connector such as a headband or clip is required for securing the electrodes to the head, and skin preparation is still required. Further, to maintain the proper electrical contact, the electrode placements must be carefully monitored to ensure that the mechanical contact is maximized and that the electrodes stay sufficiently wetted with the saline-based solution.

Each of the above-described electrodes and electrode apparatus may be utilized to monitor and record bio-potentials. The bio-potentials may be continuous, representative of normal brain activity, or may be evoked in response to an external stimuli. External stimuli may be provided to any of the sensory systems of a human body, and may include auditory stimuli, visual stimuli, olfactory stimuli, tactile stimuli, and gustatory stimuli, delivered by a suitable delivery mechanism. Conventionally, the delivery mechanism for the external stimuli is separate from the electrodes employed to measure the evoked response, requiring additional setup and handling.

Known electrode apparatus and connectors are therefore limited by being annoying or uncomfortable for the subject, especially when, placed on the head. With the additionally requirement of setting up a stimuli delivery system, the task can quickly become unduly complicated and time consuming. The discomfort or apprehension associated with the setup is a particular problem for children, infants, and uncooperative subjects. Interference by an uncooperative subject with the placement and contact of head electrodes can render recordings of evoked brain potentials impossible to obtain or useless because of minimal or inadequate contact.

A need therefore exists for an electrode apparatus configured adapted to deliver stimuli for measuring evoked potentials which is simply and comfortably positioned on the subject, which maintains adequate skin contact for obtaining measurable recordings, and which is readily and inexpensively adapted for use with small children and infants.

### SUMMARY OF THE INVENTION

An apparatus of the present invention is provided for evoking and measuring bio-potentials in a human subject. In a preferred embodiment, the apparatus includes at least one flexible member having a first surface and a shape adapted to maximize a contact area between the first surface and a selected skin surface on the human subject. At least one electrode is disposed on the first surface and positioned to make contact with the skin surface of the subject when the flexible member is disposed on the skin surface. A stimulus delivery element is coupled to the flexible member. The apparatus is used for both evoking and measuring evoked bio-potentials in the human subject, or for measuring bio-potentials evoked using a separate stimulus delivery system.

In an alternate embodiment, an apparatus is provided for evoking and measuring bio-potentials in a human subject. The apparatus includes a flexible member having a first surface and a shape adapted to maximize a contact area between the first surface and a selected skin surface of the human subject. A detector is provided for detecting evoked bio-potentials disposed on the flexible member first surface and positioned to make contact with the skin surface of the subject when the flexible member is disposed on the skin surface. A means for delivering a sensory stimulus for evoking the bio-potentials is coupled to the flexible member. The detector consists of at least one electrode, which is, for example, a layer of conductive material. The means for delivering a sensory stimulus for evoking the bio-potentials is configured for delivering either an auditory stimulus, a tactile stimulus, a gustatory stimulus, a visual stimulus, or an olfactory stimulus.

In a second alternate embodiment, the apparatus of the present invention is configured for measuring bio-potentials in a human subject and includes a flexible ear cup. The flexible ear cup defines a central space for receiving an outer ear of the subject and includes at least one surface peripheral to the central space on which an electrode is disposed to make contact with a skin surface adjacent the outer ear of the subject when the ear cup is disposed on the ear of the subject.

In a third alternate embodiment, the invention is directed toward a method of evoking and recording bio-potentials in a human subject. A flexible member is adapted to maximize a contact area between a surface of the flexible member and a selected skin surface on a human subject. At least one electrode is disposed for detecting the bio-potentials on the flexible member first surface.

The foregoing and other objects, features, and advantages of the invention as well as presently preferred embodiments thereof will become more apparent from the reading of the following description in connection with the accompanying drawings.

### BRIEF DESCRIPTION OF DRAWINGS

In the accompanying drawings which form part of the specification:

FIG. 1 is a plan view of a first side of bio-potential recording apparatus in accordance with a first embodiment of the invention;

FIG. 2 is a plan view of a second side of the apparatus shown in FIG. 1;

FIG. 3 is an plan view of a third side of the apparatus shown in FIG. 1;

FIG. 4 is a cross-sectional view of the apparatus shown in FIG. 1;

FIG. 5 is a cross-sectional view of apparatus in accordance with a second embodiment of the invention;

FIG. 6 is a plan view of a first side of the apparatus shown in FIG. 5;

FIG. 7 is a plan view of a second side of the apparatus shown in FIG. 5;

FIG. 8 is an elevational view of the apparatus shown in FIG. 5 in position on a subject's outer ear;

FIG. 9 is a perspective view of a headset including an apparatus in accordance with the embodiment shown in FIG. 1;

FIG. 10 is a perspective view of a headset including apparatus in accordance with the embodiment shown in FIG. 5;

FIG. 11 is a perspective view of an ear probe alternate embodiment of the present invention;

FIG. 12 is a sectional view of the ear probe alternate embodiment shown in FIG. 11; and

FIG. 13 is a rear view of one embodiment of the ear probe shown in FIG. 11.

Corresponding reference numerals indicate corresponding parts throughout the several Figures of the drawings.

### BEST MODE FOR CARRYING OUT THE INVENTION

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

Novel apparatus for evoking and measuring bio-potentials in a human subject and methods of use are described. In a preferred embodiment, an apparatus of the present invention includes at least one flexible member having a surface and a shape adapted to maximize a contact area between the surface and a selected skin surface on the human subject. At least one electrode is disposed on the surface and positioned to make contact with the skin surface of the subject when the flexible member is disposed on the skin surface. A stimulus delivery element is coupled to the flexible member to provide a stimulus to the human subject. The dimensions and shape of the flexible member are adapted to the specific use, i.e. type of bio-potential being measured.

More specifically, the dimensions and contours of the flexible member are adapted to conform to different surfaces on the subject's body depending on the type of bio-potential being recorded. For example, for recording auditory evoked potentials, the flexible member is adapted as a flexible ear cup that fits over the outer ear of the subject, or as an ear probe adapted for seating within the outer ear canal. For recording bio-potentials from orbital muscles attached to the eye, the flexible member is adapted as an eyecup or patch that contacts a periorbital skin surface.

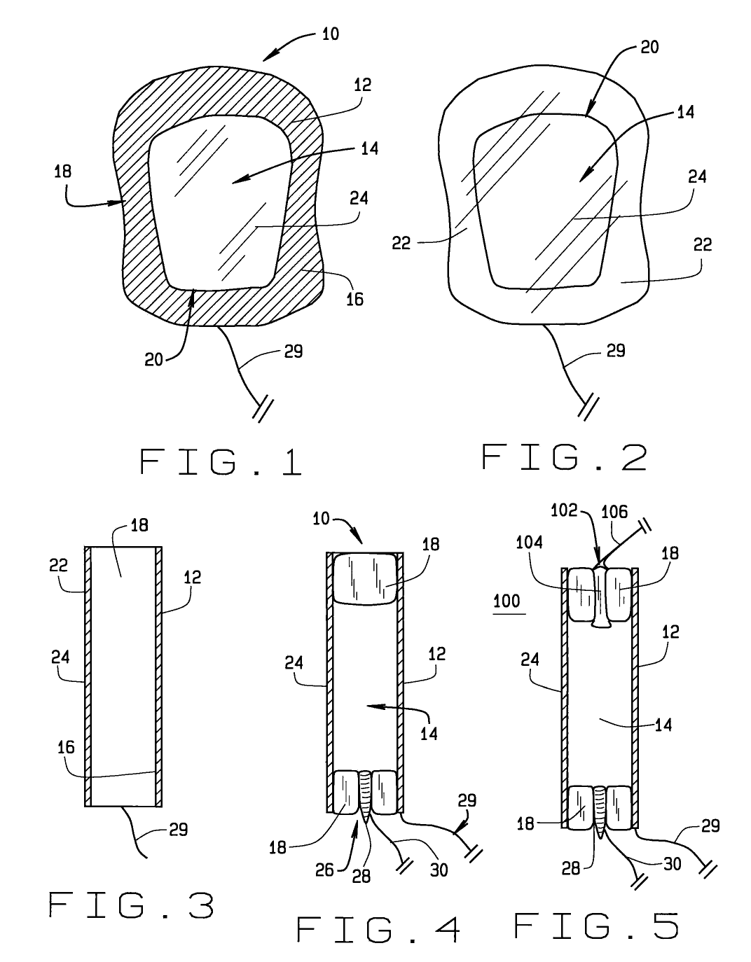

FIG. 1 is a plan view of an apparatus for recording bio-potentials in accordance with a preferred embodiment of the invention that is especially suitable for recording auditory evoked bio-potentials. An ear cup 10 has an electrode 12 disposed thereon. More specifically, ear cup 10 is a flexible ear cup defining a central space 14 for receiving an outer ear of a human subject. Ear cup 10 includes at least one surface 16 peripheral to central space 14, on which electrode 12 is disposed. Electrode 12 is positioned on surface 16 such that when the ear cup 10 is in use and in position on an outer ear of the human subject, electrode 12 makes contact with an area of skin around the outer ear of the subject.

A flexible member 18 including a central opening 20 which defines the central space 14. FIG. 2 is a plan view of a second, or outer side, of the ear cup 10 shown in FIG. 1. Flexible member 18 has an outer surface 22, on which is disposed a layer 24 of flexible material that extends across central opening 20 so that a generally cup-shape enclosure is formed, including a side wall formed by flexible member 18, and an outer wall formed by layer 24. In an exemplary embodiment, layer 24 is fabricated from a flexible and transparent plastic material so that so that in use, the outer ear is visible through layer 24.

FIG. 3 is an plan view of a third side, or edge-on view of ear cup 10 shown in FIGS. 1 and 2, in which the relative positions of electrode 12, flexible member 18, and layer 24 of flexible material are more clearly shown. Flexible member 18 has a generally annular configuration, that is, a body with a central opening therethrough. However, the precise shape of flexible member 18 need not be circular, and can be varied as desired with different shapes to accommodate different head and ear shapes.

For example, a flexible member having a semicircular, crescent shape or U-shape is also contemplated. Further, the overall dimensions of flexible member 18, the size of central opening 20, and thus also the extent of surface 16 is adaptable, to accommodate different head and ear sizes. More specifically, sizes of the elements are adapted so that the central opening receives the ear of the subject, and the flexible member fits around the outer ear of the subject so that electrode 12 on surface 16 makes contact at least with the skin over the mastoid bone of the human subject.

An ear cup 10 specifically adapted for use with infants and small children is formed in a relatively smaller size than one which would be used for adults. In particular, central space 14 and surface 16 are suitably proportioned to maintain ear cup 10 in a position that is sufficient to maintain contact of electrode 12 with an area of skin surrounding or merely adjacent to the outer ear of the subject. Thus, it will be clear that ear cups adapted for use with human infant subjects include a central space 14 and surface 16 that are suitably proportioned to maintain ear cup 10 in a position on the infant subject that is sufficient to maintain contact of electrode 12 with the area of skin surrounding or merely adjacent to the ear of the infant subject.

Flexible member 18 is fabricated from a material that can be flexibly adapted to maximize contact of electrode 12 on surface 16 with the selected area of skin, e.g. a skin surface adjacent to or surrounding the ear of the subject, such as any position along the mastoid or skull suitable for obtaining auditory evoked potentials. Any flexible material is suitable but especially suitable are resiliently compressible materials such as a closed-cell rubber material or a closed-cell synthetic foam material or the like. Other flexible materials such as rubberized materials, open-cell rubber, neoprene, stretch fabric impregnated with a rubberized material, or the like, can also be used. Also suitable are gel materials, such as a co-polymer gel or a silicone gel.

Electrode 12 is preferably formed as an annular layer of conductive material disposed on surface 16. The conductive layer defines a central opening for receiving an outer ear, wherein the central opening through the metal layer is aligned with central opening 20 through flexible member 18. Any conductive metal commonly used for surface electrodes can be used to fabricate electrode 12, and in one embodiment the conductive material is copper metal.

Alternatively, the conductive layer is a layer of conductive electrode gel or paste. The precise shape, and disposition of electrode 12 on flexible member 18 can be varied, subject only to the requirement that the conductive layer remains sufficiently pliable for the flexible member to conform to the skin surface of a human subject. More specifically, if the conductive layer is fabricated from a conductive metal such as copper metal, the metal layer must be sufficiently thin to be flexibly adjustable for maximizing the area of contact between the conductive layer and the area of skin around the outer ear of the subject. Alternatively, the relative size of electrode 12 with respect to flexible member 18 can be varied so that electrode 12 occupies only a fraction of surface 16, thus maintaining the flexibility of ear cup 10. In such a case, a relatively smaller electrode 12 is most suitably positioned on surface 16 so that in use, electrode 12 makes contact with an area of skin behind the ear, over the mastoid bone of the subject.

However, in the embodiment shown in FIGS. 1 and 2, electrode 12 is an annular layer of conductive metal disposed on surface 16 and thus configured to make contact with an annular-shaped area of skin around the outer ear of the subject. In another alternative embodiment, multiple electrodes are arranged in an array across surface 16, wherein each of the multiple electrodes occupies a fraction of the area of surface 16 and the array of electrodes includes sufficient area between each electrode such that the flexibility of the flexible member is maintained. In such an embodiment, it will be understood that each individual electrode need not itself be flexible.

While the apparatus is adaptable for recording any bio-potentials, ear cup 10 as shown in FIGS. 1-8 is especially suitable for recording auditory evoked potentials in response to auditory stimuli, such as, but not limited to, the auditory brainstem response (ABR). FIG. 4 is a cross-sectional view of ear cup 10, showing an opening 26 through a portion of flexible member 18, for receiving a miniature speaker 28 with a lead 30 for controlled introduction of auditory stimuli, such as electronically generated tones, to the ear of the human subject.

Optionally, the miniature speaker 28 may be disposed remotely from the ear cup 10, and operatively coupled thereto via a tube or other acoustic wave guide. This would permit a single miniature speaker 28 to be disposed remotely from the patient, and provide auditory stimuli to each ear of the patient simultaneously through an interconnecting tube or wave guide.

In alternative embodiments adapted for evoking and measuring other types of bio-potentials, other types of stimulus delivery elements are used in accordance with the type of bio-potential being measured. Visual stimulus may be provided using a light source such as an LED or multiple LED's. Tactile stimulus may be provided by a needle or textured surface. Olfactory stimulus may be provided by a nozzle or pipette configured to deliver a puff of gas or aerosolized scent, and gustatory stimulus may be provided by a mouthpiece, straw, or tube configured for delivering a sample of a liquid to be tasted.

In use for measuring bio-potentials, an electrode lead or leads 29 is secured to electrode 12 for example by a metal clip or solder, and the lead or leads are coupled to suitable data acquisition equipment as known, such as an amplifier and computer with software configured for real-time data acquisition. The ear cups are especially useful in combination with computerized auditory screening devices that use the ABR signal as a basis for determining hearing loss in infants, such as the AUDIOscreener™ commercially available from Everest Biomedical Instruments Company of Chesterfield, Mo.

An ear cup 10 is fitted over each ear of the subject and manually adjusted, taking advantage of the flexibility of ear cup 10 so that electrode 12 makes adequate contact with an area of skin surrounding, or adjacent to, the ear. A third, ground electrode such as a disc electrode, with a ground lead, is secured to the forehead of the subject and the lead coupled to the data acquisition set-up. Spontaneous brain activity can then be recorded. For evoked potentials, a stimulus or series of stimuli is introduced, and resulting bio-potentials are detected by the electrodes. As explained above, auditory stimuli for evoking auditory responses, such as a series of tones or tone bursts, are introduced through speaker 26 and the resulting auditory evoked potentials, including ABR are recorded.

In an alternate embodiment of the ear cup, a second opening through flexible member 18 is provided for receiving a miniature microphone for recording evoked otoacoustic emissions (OAF). FIG. 5 is a cross-sectional view of an ear cup 100 with an electrode 12, showing a second opening 102 though flexible member 18, and a miniaturized microphone 104 disposed within opening 102. The miniaturized microphone 104 is disposed with its receiving end directed toward central space 14, to record OAE. A lead 106 from microphone 104 is coupled to the data acquisition equipment, such as an AUDIOscreener™ as described above.

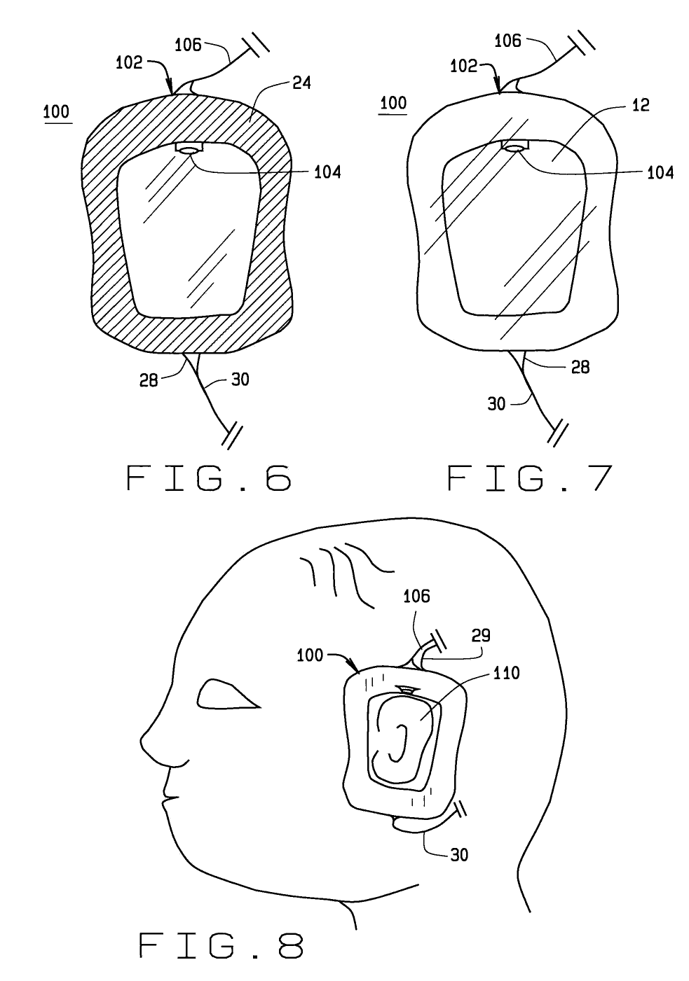

Auditory stimuli are introduced through speaker 26 and the resulting auditory evoked potentials, including ABR as well as OAE are recorded and analyzed to determine hearing loss. FIG. 6 is a plan view of a first side of the ear cup shown in FIG. 5, and FIG. 7 is a plan view of a second side of the ear cup shown in FIG. 5, both showing the disposition of microphone 104 within opening 102 through flexible member 18.

FIG. 8 is a perspective view of the ear cup shown in FIG. 5 showing the disposition of the ear cup on a human subject's ear 110 when in use. An electrode lead 29 coupled to electrode 12, lead 30 from speaker 28, and lead 106 from microphone 104 are routed to the data acquisition equipment (not shown). As described above, a second like ear cup 10 is positioned over the subject's other outer ear, and a third, ground electrode positioned on the forehead of the subject. Not visible in FIG. 8 is electrode 12, which in use, abuts an area of skin surrounding the ear, and maintains contact with at least an area of skin over the mastoid bone, behind the ear.

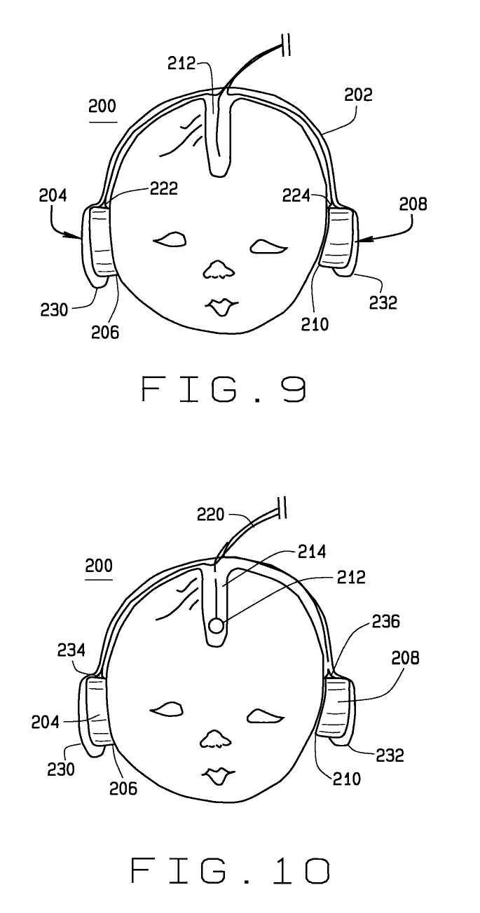

The invention is further directed towards a headset with ear cups for recording bio-potentials in a human subject. FIG. 9 is a perspective view of a headset 200 including a pair of ear cups 10 as shown and described in FIGS. 1-4 and corresponding text. FIG. 10 is a perspective view of a headset including a pair of ear cups 100 as shown and described in FIGS. 5-7 and corresponding text.

As shown in FIG. 9, headset 200 includes ear cups 10 without a microphone 104, suitable for applications requiring detection of brain bio-potentials but not for applications requiring detection of OAE. Headset 200 includes a pair of ear cups 10 disposed on a flexible headband 202 adapted to fit over the head of the subject so that a first ear cup 204 with a first ear cup electrode 206 receives a first outer ear (not visible in FIG. 9) of the human subject, and a second ear cup 208 with a second ear cup electrode 210 receives a second outer ear (not visible in FIG. 9) of the human subject.

In an exemplary embodiment, the flexible headband is fabricated from a resiliently flexible plastic material and is adjustable in size to adapt to different head sizes, particularly the dimensions of the head across the crown of the head top from ear to ear. Alternatively, headband 202 may be fabricated from any suitable resiliently flexible material including a vinyl, rubber or rubberized material, or any combination thereof. As used herein, the term “resiliently flexible” refers to the characteristic of a material that allows the material to be bent from a starting configuration without breaking, and then to return to the starting configuration. The size adjustment is achieved, for example by having two separate but slidably engaged elements that are manually positioned with respect to one another as is well known in the art of lightweight plastic headphones.

For example, each ear cup 10 may simply mounted on each end of headband 202 using an adhesive to bond the end of headband 202 to flexible layer 24. However, any number of attachment means might also be used to couple ear cup 10 to headband 200. For example, using releasable attachments for each ear cup 10 permits either the ear cups 10 or the headband 202 to be removed for replacement or discarding. Optionally, the ear cups 10 may be secured to the patient using a releasable medical adhesive, eliminating the need for a headband 202.

A ground electrode 212 is disposed on a central support member 214 that extends from the flexible headband 202. Central support member 214 is configured to maintain contact between ground electrode 212 and an area of skin on the forehead of the subject. While multiple configurations will satisfy this last requirement, an exemplary embodiment includes central support member 214 extending anteriorly with respect to the subject's head, and in a downward curve that brings electrode 212 into contact with an area of skin on the subject's forehead. Electrode 212 is secured, for example, with adhesive, or with a detachable connector, to the undersurface of central support member 214, and positioned to make contact with an area of skin on the subject's forehead.

In an alternative embodiment, as shown in FIG. 10, electrode 212 is secured to support member 214 over an opening through central support member 214 that permits electrode 212 to make skin contact through the opening. Alternative configurations of electrode 212 with respect to support member 214 are also contemplated within the scope of the present invention, subject to the requirement that electrode 212 is suitably positioned for support member 214 to maintain electrode 212 in adequate contact with the skin of the human subject.

Headset 200 further includes a lead or cable 220 for electronically coupling first ear cup electrode 208, second ear cup electrode 210 and ground electrode 212 to biofeedback monitoring apparatus. In accordance with the ear cup shown in FIGS. 1-3, the ear cups in one embodiment of headset 200, as shown in FIG. 9, each include, a speaker disposed within a speaker cavity in the annular member. Further, cable 220 includes multiple electrical leads for coupling each of the electronic elements to suitable biofeedback monitoring apparatus. More specifically, such leads include a first ear cup electrode lead 222 coupled to first ear cup electrode 204, a second ear cup electrode lead 224 coupled to second ear cup electrode 208, a ground lead 226 coupled to ground electrode 212, a first speaker lead 230 coupled to the first ear cup speaker and a second speaker lead 232 coupled to the second ear cup speaker.

In the alternative embodiment of headset 200, as shown in FIG. 10 and including the ear cups as shown in FIGS. 4-7, each ear cup 10 further includes a microphone disposed within a microphone cavity in ear cup annular member. Accordingly, a first microphone lead 234 is coupled to the first ear cup microphone, a second microphone lead 236 is coupled to the second ear cup microphone and these leads further coupled, through cable 220 to suitable biofeedback monitoring apparatus.

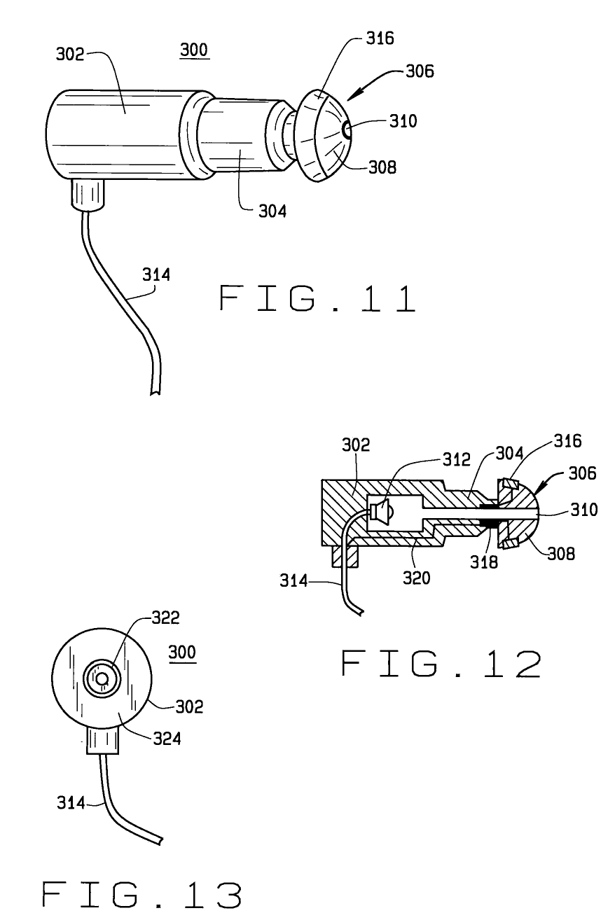

In an additional embodiment of the present invention, shown in FIG. 11, provides an internal ear probe 300 configured for evoking and measuring bio-potentials in a human subject. The ear probe 300 consists of a cylindrical body 302, and an elongated neck portion 304. A disposable ear canal plug 306 is fitted to the elongated neck portion 304, opposite the cylindrical body 302. The ear canal plug 306 include a hemispherical shaped head 308, sized to seat within the ear canal of a human subject. The hemispherical shaped head 308 preferably consists of a flexible material, such as rubber or urethane, which will confirm to the inner skin surfaces of an ear canal in a human subject. An opening 310 is axially disposed in the head 308, for conveying auditory stimuli transmitted through the elongated neck portion 304 from a miniature speaker 312 contained within the cylindrical body 302, as seen in FIG. 12. Miniature speaker 312 is coupled to a lead 314 to the data acquisition equipment, such as an AUDIOscreener™ as described above, for controlled the introduction of auditory stimuli, such as electronically generated tones, to the ear of the human subject.

Bio-potentials generated by the human subject in response to the auditory stimuli are received by an electrode 316 preferably formed as an annular layer of conductive material disposed on the hemispherical shaped head 308. The conductive layer Any conductive metal commonly used for surface electrodes can be used to fabricate electrode 316, and in one embodiment the conductive material is copper metal. Electrode 316 is disposed on the hemispherical shaped head 308 such that when the ear probe 300 is seated within the ear canal of a human subject, the electrode 316 is in operative contact with skin surfaces to receive and detect bio-potentials generated in response to the auditory stimuli. Signals from the electrode 316 are routed through detachable contacts 318 between the hemispherical shaped head 308 and the elongated neck portion 304 of the ear plug 300, and conveyed via internal an internal lead 320 to the data acquisition equipment for subsequent processing. Those of ordinary skill in the art will recognize that the hemispherical shaped head 308, and associated electrode 316 are configured to be removed from the elongated neck portion 304, for disposal and replacement.

In an alternate configuration, shown in FIG. 13, the ear plug 300 is not configured for use with an electrode 316 disposed on the hemispherical shaped head 308. Rather, a lead coupling 322 is disposed in the rear face 324 of the cylindrical body 302 and adapted to receive a lead from an external disposable electrode (not shown). The external disposable electrode may then be placed in any suitable location on the skin of the human subject, adjacent the ear within which the ear probe 300 is seated. Internal leads (not shown) routed along lead 314 link the lead coupling 322 to the data acquisition equipment, and permit signals received from an external disposable electrode to be conveyed thereto.

As a method of providing electrodes for measuring bio-potentials in a human subject, at least a first electrode is disposed on a first flexible ear cup. The first ear cup is positioned on a human subject to receive a first outer ear of the human subject and to maintain a position on the outer ear sufficient to maintain contact of the first electrode with a skin surface adjacent to the first outer ear. Depending on the age and cooperativeness of the subject and type of bio-potential being measured, a second electrode is also provided disposed on a second flexible ear cup. The second ear cup disposed to receive a second outer ear of the subject and to maintain a position of the second ear cup on the second outer ear sufficient to maintain contact of the second electrode with a skin surface adjacent to the second outer ear. Alternatively, the second electrode and ground electrode are disposed as conventional surface electrodes on the skin of the human subject, and are not disposed on a second ear cup.

Alternatively, the first ear cup and second ear cup are provided disposed on a flexible headband as described above, and the headband is fitted over the head of a human subject such that the first ear cup receives a first outer ear of the human subject and the second ear cup receives a second outer ear of the human subject. A ground electrode is provided on a central support member as described above, and brought into contact with the human subject's forehead skin. Alternatively, a ground electrode separate and apart from the headband is secured in a conventional manner to skin on the forehead.

During use, each ear cup 10 is fitted over an outer ear of the human subject so that contact is maintained between the electrode and the skin surface. The flexibility of the ear cup flexible member is used advantageously to adjust the ear cup to conform to the subject's surrounding bone structure. A stimulus, such as an audible tone or pulse, is provided to the human subject, through first speaker disposed on the first ear cup and a second speaker disposed on the second ear cup. Bio-potential responses from the human subject are detected through the electrodes. Alternatively, oto-acoustic emissions evoked in response to the stimulus may be recorded via first and second microphones disposed in the first and second ear cups.

In view of the above, it will be seen that the several objects of the invention are achieved and other advantageous results are obtained. As various changes could be made in the above constructions without departing from the scope of the invention, it is intended that all matter contained in the above description or shown in the accompanying drawings shall be interpreted as illustrative and not in a limiting sense.

1-19. (canceled)

20. A headset for measuring bio-potentials in a human subject, comprising
two ear cups each defining a central space for receiving an outer ear of the human subject with an annular surface peripheral to the central space adapted to conform to a skin surface adjacent an outer ear of the human subject, and
an electrode disposed on the annular surface configured to conform to the skin surface and to detect bio-potential signals through the skin surface in the human subject, disposed on a flexible headband adapted to fit over the head of the human subject and to position a first ear cup with a first electrode to receive a first outer ear of the human subject, and to position a second ear cup with a second electrode to receive a second outer ear of the human subject.

21. A headset in accordance with claim 20 wherein said flexible headband comprises a size-adjustable headband for adapting the headset to the head size of the human subject.

22. A headset in accordance with claim 21 further including a ground electrode disposed on a support member extending from said flexible headband, said support member configured to maintain contact between said ground electrode and a skin surface on the forehead of the human subject.

23. A headset in accordance with claim 22 further comprising a cable for electronically coupling said first ear cup electrode, said second ear cup electrode, and said ground electrode to a data acquisition apparatus.

24. A headset in accordance with claim 23 wherein said first ear cup includes a first speaker disposed within a first speaker cavity in said first ear cup annular member, and said second ear cup includes a second speaker disposed within a second speaker cavity in said second ear cup annular member.

25. A headset in accordance with claim 24 wherein said cable comprises a first ear cup electrode lead coupled to said first ear cup electrode, a second ear cup electrode lead coupled to said second ear cup electrode, a ground lead coupled to said ground electrode, a first speaker lead coupled to said first speaker and a second speaker lead coupled to said second speaker.

26. A headset in accordance with claim 23 wherein said first ear cup includes a microphone disposed within a first microphone cavity in said first ear cup annular member, and said second ear cup includes a second microphone disposed within a second microphone cavity in said second ear cup annular member.

27. A headset in accordance with claim 26 wherein said cable further comprises a first ear cup microphone lead coupled to said first ear cup microphone and a second ear cup microphone lead coupled to said second ear cup microphone.

28-33. (canceled)

34. A headset for evoking and detecting bio-potentials in a human subject, comprising:
a first flexible member having a first surface and a shape adapted to contact a first selected skin surface on the human subject;
a first stimulus delivery element disposed in operative relation to said first flexible member, said first stimulus delivery element configured to provide a stimulus adapted to evoke a bio-potential in said human subject;
at least one bio-potential detector disposed on said first surface to contact said first selected skin surface, said at least one bio-potential detector configured to detect said evoked bio-potential from said first selected skin surface;
a second flexible member having a second surface and a shape adapted to contact a second selected skin surface on the human subject;
a second stimulus delivery element disposed in operative relation to said second flexible member, said second stimulus delivery element configured to provide a stimulus adapted to evoke said bio-potential in said human subject;
at least one bio-potential detector disposed on said second surface to contact said second selected skin surface, said at least one bio-potential detector configured to detect an evoked bio-potential from said second selected skin surface;
a flexible headband adapted to fit over a head of the human subject and to support said first and second flexible members in operative relation to said first and second selected skin surfaces, respectively; and
wherein said first and second selected skin surfaces are respectively disposed within first and second ear canals of the human subject.

35. (canceled)

36. A headset for evoking and detecting bio-potentials in a human subject, comprising:
a pair of ear cups, each ear cup defining a central space for receiving an outer ear of the human subject, each said ear cup comprising at least one annular surface peripheral to said central space, said annular surface adapted to conform to a skin surface adjacent the outer ear of the human subject;
an electrode disposed on said annular surface of each of said ear cups, said electrodes configured to conform to said skin surface and to detect bio-potential signals through said skin surface;
a microphone disposed within a microphone cavity in said annular surfaces of each of said pair of ear cups;
an audio speaker disposed within a speaker cavity in said annular surfaces of each of said pair of ear cups; and
a flexible headband adapted to fit over the head of the human subject to position each of said ear cups and electrodes to receive an outer ear of the human subject.
